(19) United States
(12) Patent Application Publication
(10) Pub. No.: US 2013/0189246 A1
(43) Pub. Date: Jul. 25, 2013

(54) TREATMENT OF OPHTHALMIC CONDITIONS WITH FLUORENONE DERIVATIVES

(71) Applicant: Konstantin Petrukhin, New York, NY (US)

(72) Inventor: Konstantin Petrukhin, New York, NY (US)

(73) Assignee: The Trustees of Columbia University in the City of New York, New York, NY (US)

(21) Appl. No.: 13/509,712

(22) PCT Filed: Nov. 5, 2010
(86) PCT No.: PCT/US10/55540

§ 371 Date: Feb. 25, 2013

Related U.S. Application Data
(60) Provisional application No. 61/260,439, filed on Nov. 12, 2009.
(60) Provisional application No. 61/378,624, filed on Aug. 31, 2010.

(51) Int. Cl. A61K 31/192; C07D 263/14; C07D 277/10; A61K 45/06; A61K 31/535; A61K 31/421; A61K 31/426; A61K 31/44; C07D 265/06; C07D 213/65

(52) U.S. Cl. CPC A61K 31/192; C07D 265/06; C07D 263/14; C07D 277/10; C07D 213/65; A61K 31/535; A61K 31/421; A61K 31/426; A61K 31/44; A61K 45/06
USPC 424/133.1; 514/569; 514/228.8; 514/374; 514/365; 514/345; 424/94.64; 514/29; 514/44 A; 514/171; 514/20.5; 514/45

(57) ABSTRACT

Provided are compositions and methods for treatment of ophthalmic conditions, such as retinal detachment and age-related macular degeneration. Various fluorenone derivatives described herein can stimulate fluid removal from the subretinal space and down-regulate reactive gliosis. Administration of compounds described herein can provide an alternative or an adjunct to an invasive procedure to reattach the retina.

### CROSS-REFERENCE TO RELATED APPLICATIONS

The present application claims the benefit of U.S. Provisional Application Ser. No. 61/260,439 filed 12 Nov. 2009; and U.S. Provisional Application Ser. No. 61/378,624 filed 31 Aug. 2010; all of which are incorporated herein by reference in their entireties.

### STATEMENT REGARDING FEDERALLY SPONSORED RESEARCH OR DEVELOPMENT

Not applicable.

### MATERIAL INCORPORATED-BY-REFERENCE

Not Applicable.

### FIELD OF THE INVENTION

The present invention generally relates to treatment of ophthalmic conditions.

### BACKGROUND

Retinal detachment is a potentially blinding condition affecting approximately one in 300 patients in the course of a lifetime. Anatomically, retinal detachment represents separation of the neural retina from the retinal pigment epithelium (RPE) and accumulation of fluid in the subretinal space. In addition, retinal detachment is characterized by profound reactive gliosis in the retina.

There are two general classes of retinal detachment, rhegmatogenous and non-rhegmatogenous. Conventional treatment for the two main classes (rhegmatogenous vs non-rhegmatogenous) is different because the etiologies are different.

In rhegmatogenous (more common), there is disruption or tearing of the retina which leads to leakage of fluid/vitreous into the subretinal space. This is typically seen in cases of trauma, posterior vitreous detachment (PVD) or high myopia and prior cataract surgery. Incidence of non-traumatic rhegmatogenous retinal detachment is 1:10,000 in the general population. This is the more common form compared to non-rhegmatogenous or exudative retinal detachment. The current standard of care for rhegmatogenous RD is surgical intervention with pneumatic retinopexy, scleral buckling techniques or vitrectomy to repair the tear and reattach the retina. Surgery for uncomplicated cases is up to 1.5 hrs in duration and has an anatomical success rate of >90% and an overall success of 60-95%. Yet, even after surgery, 40% of patients will not achieve reading ability and 10-40% will need another procedure. Complications of surgery include pain, hemorrhage infection, buckle extrusion, lens trauma, cataract progression, and proliferative vitreoretinopathy (PVR), which generally occurs in less than 5% of patients. The cost of surgery (scleral buckling or vitrectomy) can range from $1400-$2500. Sophisticated procedure of surgical reattachment is expensive and may be sparsely available as it is performed only at tertiary health care centers.

In the case of non-rhegmatogenous retinal detachment, proteinaceous exudate leaks into the space between the RPE and neural retina leading to the detachment. This is typically seen in inflammatory conditions (e.g., chorioretinopathy) or choroidal tumors, which may cause a breakdown of the blood-retina barrier or impaired fluid flow in the subretinal space. Because there is usually no hole or tear to be repaired, surgery is not the mainstay of therapy for non-rhegmatogenous retinal detachment. For non-rhegmatogenous retinal detachment due to exudates, the underlying cause of the exudative fluid (e.g., inflammation, diabetic retinopathy, tumor) must be treated to eliminate the exudative process. Occasionally, thermotherapy or cryotherapy and intravitreal steroids are used to reattach the retina and prevent gliosis, edema or inflammation.

Intravitreal injections of gases during pneumatic retinopexy has been used for retinal detachment but there are no FDA approved medications to treat retinal detachment via intravitreal injection. Intravitreal injections is a short office based procedure that can be done under topical or local anesthesia. Complications can include pain, subretinal hemorrhage, subconjunctival hemorrhage or temporary elevation of IOP.

Age-related macular degeneration (AMD) is the leading cause of blindness in developed countries. There is no effective treatment for the most prevalent atrophic (dry) form of AMD. Atrophic AMD is thought to be triggered by abnormalities in the retinal pigment epithelium (RPE) that lies beneath the photoreceptor cells and normally provides critical metabolic support to these light-sensing cells. Secondary to RPE dysfunction, macular rods and cones degenerate leading to the irreversible loss of vision. Oxidative stress, formation of drusen, accumulation of lipofuscin, local inflammation and reactive gliosis are thought to represent the pathologic processes implicated in pathogenesis of atrophic AMD.

### SUMMARY OF THE INVENTION

Among the various aspects of the present invention is the provision of compositions and methods for treatment of ophthalmic conditions.

One aspect provides a method for treating an ophthalmic condition. A pharmaceutical composition comprising an effective amount of a fluorenone derivative is administered to a subject in need thereof. In some embodiments, the fluorenone derivative is a compound of formula (1).

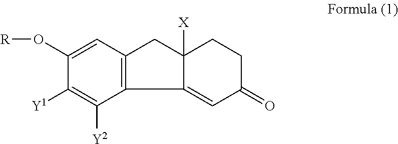

In Formula (1), X is selected from the group consisting of lower alkyl containing 1 to 3 carbon atoms; substituted lower alkyl; and lower cycloalkyl; R is a substituted alkyl group in which the substituents are selected from the group consisting of aryl and substituted aryl; and substituted or unsubstituted heterocyclic rings having 0 or 1 nitrogen atom and at least one double bond wherein the alkyl group is attached to a carbon atom of the heterocyclic ring; and Y¹ and Y² are independently selected from the group consisting of halogen, hydrogen, and methyl.

In some embodiments, the ophthalmic condition is selected from the group consisting of retinal detachment or age-related macular degeneration (AMD). In some embodiments, the ophthalmic condition is retinal detachment. In some embodiments, the ophthalmic condition is AMD. In some embodiments, the ophthalmic condition is atrophic (dry) AMD or neovascular (wet) AMD. In some embodiments, the ophthalmic condition is atrophic (dry) AMD. In some embodiments, the ophthalmic condition is neovascular (wet) AMD.

In some embodiments, X is selected from the group consisting of propyl, hydroxyethyl, haloethyl, and cycloalkyl having less than 6 carbons. In some embodiments, R is a heterocyclic-alkyl group. In some embodiments, R is an oxazinyl-alkyl group. In some embodiments, the compound is selected from the group consisting of: 2-{[(5,6-dichloro-2,3,9,9a-tetrahydro-3-oxo-9a-propyl-1H-fluoren-7-yl)oxy]methyl}-tetrahydro-1,3-oxazine; 2-{[(5,6-dichloro-2,3,9,9a-tetrahydro-3-oxo-9a-propyl-1H-fluoren-7-yl)oxy]methyl}oxazoline; 2-{[(5,6-dichloro-2,3,9,9a-tetrahydro-3-oxo-9a-propyl-1H-fluoren-7-yl)oxy]methyl}thiazoline; and enantiomers thereof; and pharmaceutically acceptable salts thereof.

In some embodiments, R is a pyridyl-alkyl group. In some embodiments, the compound is selected from the group consisting of: 5,6-dichloro-9a-propyl-7-(2-pyridylmethoxy)-2,3,9,9a-tetrahydro-1H-fluoren-3-one; 5,6-dichloro-9a-propyl-7-(3-pyridylmethoxy)-2,3,9,9a-tetrahydro-1H-fluoren-3-one; 5,6-dichloro-9a-propyl-7-(4-pyridylmethoxy)-2,3,9,9a-tetrahydro-1H-fluoren-3-one; and enantiomers thereof; and pharmaceutically acceptable salts thereof.

In some embodiments, R is a heterocyclicaralkyl group. In some embodiments, the compound is selected from the group consisting of: 5,6-dichloro-2,3,9,9a-tetrahydro-7-[4-(2-oxazolinyl)-phenylmethoxy]-9a-propyl-1H-fluoren-3-one; 5,6-dichloro-2,3,9,9a-tetrahydro-7-[3-(2-oxazolinyl)-phenylmethoxy]-9a-propyl-1H-fluoren-3-one; 5,6-dichloro-2,3,9,9a-tetrahydro-7-[2-(2-oxazolinyl)-phenylmethoxy]-9a-propyl-1H-fluoren-3-one; and, where applicable, enantiomers thereof; and pharmaceutically acceptable salts thereof.

In some embodiments, X is propyl; R is carboxymethyl; and Y¹ and Y² are chorine. In some embodiments, the compound is [(R)-(+)-(5,6-dichloro 2,3,9,9a-tetrahydro 3-oxo-9a-propyl-1H-fluoren-7-yl)oxy]acetic acid (DPOFA), having a structure as follows:

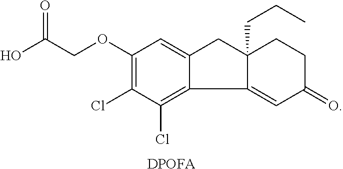

In some embodiments, the compound is a prodrug.

In some embodiments, the subject is a mammal. In some embodiments, the subject is selected from the group consisting of a human, monkey, horse, cow, dog, cat, sheep, pig, mice, rat, guinea pig, and chicken.

In some embodiments, the composition is administered ophthalmically. In some embodiments, the administration comprises subscleral, subtenon, subconjunctival, intravitreal, or topical administration. In some embodiments, the administration comprises intravitreal injection.

In some embodiments, the composition further comprises an ophthalmic agent selected from the group consisting of an ophthalmic dye, an ophthalmic anesthetic, an ophthalmic mydriatic, an ophthalmic cycloplegic mydriatic, an ophthalmic anticholinergic, and ophthalmic anti-inflammatory, an ophthalmic corticosteroid, ophthalmic artificial tears or lubricants, an ophthalmic antibiotic, an ophthalmic antifungal, an ophthalmic antiviral, an ophthalmic epinephrine, an ophthalmic beta blocker, an ophthalmic surgical adjunct, an ophthalmic intraocular irrigant, and an ophthalmic viscoelastic agent.

In some embodiments, the ophthalmic agent is selected from the group consisting of: Acular (ketorolac tromethamine), AK-Con-A (naphazoline ophthalmic), Akten (lidocaine hydrochloride), Alamast, Alphagan (brimonidine), Alrex, Avastin (bevacizumab), Atropine, AzaSite (azithromycin), Azopt, Bacitracin, Betadine, Betaxolol, Betaxon, Betoptic, Brinzolamide, BSS, Carbachol, Cefazolin, Celluvisc, Chloramphenicol, Ciloxan, Ciprofloxacin, Cosopt, Demecarium, Denufosol tetrasodium, Dexamethasone, Dipivefrin, Dorzolamide, Durezol (difluprednate), Epinephrine, Fluorescein, Flurbiprofen, Gentamicin, Goniosol, Gramicidin, Humorsol, Hylartin, Hypertonic NaCl, Indocycanine Green, Itraconazole, Latanoprost, Lotemax, Lucentis (ranibizumab), Lumigan (bimatoprost ophthalmic solution), Macugen (pegaptanib), Mannitol, Methazolamide, Miconazole, Miostat, Muro 128, Neomycin, Neptazane, Ocuflox, OcuHist, Ofloxacin, Oxytetracycline, Palomid 529, Phenylephrine, Physostimine, Pilocarpine, Plasmin enzyme, Polymyxin B, Prednisolone, Proparacaine, Propine, Puralube, Quixin (levofloxacin), Rescula (unoprostone isopropyl ophthalmic solution), Restasis (cyclosporine ophthalmic emulsion), Rose Bengal, sodium hyaluronate, Suprofen, Terramycin, Timolol, Tobramycin, Triamcinolone, Trifluridine, Tropicamide, Trusopt, Valcyte (valganciclovir HCl), Vidarabine, Vira-A, Viroptic, Vistide (cidofovir injection), Visudyne (verteporfin for injection), Vitrase (hyaluronidase), Vitrasert Implant, Vitravene Injection, Xalatan, and Zaditor.

In some embodiments, the pharmaceutical composition comprises a pharmaceutically acceptable carrier or excipient. In some embodiments, the pharmaceutical composition comprises silicon oil.

In some embodiments, the method further comprises monitoring the subject for symptoms of the ophthalmic condition. In some embodiments, the method further comprises monitoring the subject for symptoms of retinal detachment or side effects of the procedure. In some embodiments, the method further comprises monitoring the subject for one or more of: retinal re-detachment, hemorrhage, infection, buckle extrusion, lens trauma, cataract progression, and proliferative vitreoretinopathy. In some embodiments, the method further comprises monitoring the subject for symptoms of AMD. In some embodiments, the method further comprises monitoring the subject for one or more of: drusen, pigmentary alterations, exudative changes, atrophy, decreased visual acuity, preferential hyperacuity perimetry changes, blurred vision, central scotomas, metamorphopsia, difficulty discerning colors, slow recovery of visual function after exposure to bright light, or a loss in contrast sensitivity.

In some embodiments, the method comprises re-administering the composition. In some embodiments, the method comprises re-administering the composition according to results of monitoring the subject.

One aspect provides a pharmaceutical composition comprising a fluorenone derivative, an ophthalmic agent, and a pharmaceutically acceptable carrier or excipient.

In some embodiments, the fluorenone derivative is a compound of Formula (1).

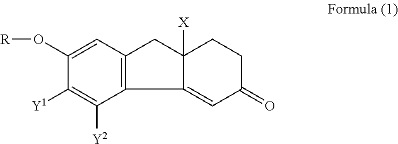

According to Formula (1), X is selected from the group consisting of lower alkyl containing 1 to 3 carbon atoms; substituted lower alkyl; and lower cycloalkyl; R is a substituted alkyl group in which the substituents are selected from the group consisting of aryl and substituted aryl; and substituted or unsubstituted heterocyclic rings having 0 or 1 nitrogen atom and at least one double bond wherein the alkyl group is attached to a carbon atom of the heterocyclic ring; and Y¹ and Y² are independently selected from the group consisting of halogen, hydrogen, and methyl. In some embodiments of the pharmaceutical composition, substituents of Formula (1) can be any of those discussed above.

In some embodiments of the pharmaceutical composition, the fluorenone derivative is [(R)-(+)-(5,6-dichloro 2,3,9,9a-tetrahydro 3-oxo-9a-propyl-1H-fluoren-7-yl)oxy]acetic acid (DPOFA), having a structure as follows:

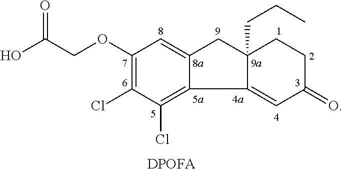

In some embodiments, the ophthalmic agent is an ophthalmic dye, and ophthalmic anesthetic, an ophthalmic mydriatic, an ophthalmic cycloplegic mydriatic, an ophthalmic anticholinergic, and ophthalmic anti-inflammatory, an ophthalmic corticosteroid, ophthalmic artificial tears or lubricants, an ophthalmic antibiotic, an ophthalmic antifungal, an ophthalmic antiviral, an ophthalmic epinephrine, an ophthalmic beta blocker, an ophthalmic surgical adjunct, an ophthalmic intraocular irrigant, or an ophthalmic viscoelastic agent.

In some embodiments, the ophthalmic agent is selected from the group consisting of: Acular (ketorolac tromethamine), AK-Con-A (naphazoline ophthalmic), Akten (lidocaine hydrochloride), Alamast, Alphagan (brimonidine), Alrex, Atropine, Avastin (bevacizumab), AzaSite (azithromycin), Azopt, Bacitracin, Betadine, Betaxolol, Betaxon, Betoptic, Brinzolamide, BSS, Carbachol, Cefazolin, Celluvisc, Chloramphenicol, Ciloxan, Ciprofloxacin, Cosopt, Demecarium, Denufosol tetrasodium, Dexamethasone, Dipivefrin, Dorzolamide, Durezol (difluprednate), Epinephrine, Fluorescein, Flurbiprofen, Gentamicin, Goniosol, Gramicidin, Humorsol, Hylartin, Hypertonic NaCl, Indocycanine Green, Itraconazole, Latanoprost, Lotemax, Lucentis (ranibizumab), Lumigan (bimatoprost ophthalmic solution), Macugen (pegaptanib), Mannitol, Methazolamide, Miconazole, Miostat, Muro 128, Neomycin, Neptazane, Ocuflox, OcuHist, Ofloxacin, Oxytetracycline, Palomid 529, Phenylephrine, Physostimine, Pilocarpine, Plasmin enzyme, Polymyxin B, Prednisolone, Proparacaine, Propine, Puralube, Quixin (levofloxacin), Rescula (unoprostone isopropyl ophthalmic solution), Restasis (cyclosporine ophthalmic emulsion), Rose Bengal, sodium hyaluronate, Suprofen, Terramycin, Timolol, Tobramycin, Triamcinolone, Trifluridine, Tropicamide, Trusopt, Valcyte (valganciclovir HCl), Vidarabine, Vira-A, Viroptic, Vistide (cidofovir injection), Visudyne (verteporfin for injection), Vitrase (hyaluronidase), Vitrasert Implant, Vitravene Injection, Xalatan, and Zaditor.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a line and scatter plot showing the increase in meniscus position of the basal capillary (microliters) as a function of time (minutes) in a first experiment using bovine RPE-choroid before, during and after treatment with 6 μM, 20 μM, and 60 μM DPOFA. Further methodology information is according to Example 1.

FIG. 2 is a line and scatter plot showing the increase in meniscus position of the basal capillary (microliters) as a function of time (minutes) in a second experiment using bovine RPE-choroid before, during and after treatment with 6 μM, 20 μM, and 60 μM DPOFA. Further methodology information is according to Example 1.

### DETAILED DESCRIPTION OF THE INVENTION

Described herein include compositions and methods for treatment of ophthalmic conditions, such as retinal detachment and age-related macular degeneration (AMD). Various aspects are based, at least in part, on the discovery that fluorenone derivative [(R)-(+)-(5,6-dichloro 2,3,9,9a-tetrahydro 3-oxo-9a-propyl-1H-fluoren-7-yl)oxy]acetic acid (DPOFA) can effectively stimulate water removal across the retinal pigment epithelium.

Some embodiments of the pharmacological compounds and protocols described herein for the treatment for ophthalmic conditions can combine two distinct activities: stimulation of fluid removal from the subretinal space and down-regulation of reactive gliosis. Given that the standard therapy for retinal detachment is surgery, methods described herein can provide an alternative or an adjunct to an invasive procedure to reattach the retina. Various pharmacological treatments described herein can be administered by a general ophthalmologist, making these treatments widely available.

Fluorenone Derivatives

A fluorenone derivative can be used for the treatment of ophthalmic conditions, such as retinal detachment and AMD. In some embodiments, the fluorenone derivative is a broadly-specific small molecule Cl⁻/HCO₃⁻ antiporter inhibitor. In some embodiments, the fluorenone derivative can stimulate fluid removal from the subretinal space. In some embodiments, the fluorenone derivative can down-regulate reactive gliosis. In some embodiments, the fluorenone derivative can stimulate fluid removal from the subretinal space and down-regulate reactive gliosis.

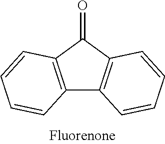 [SMILES: O=C1c2ccccc2-c2ccccc21]

Exemplary fluorenone derivatives for use in compositions and methods described herein include, but are not limited to, those compounds described in U.S. Pat. No. 6,251,898; WO2001/014334; U.S. Pat. No. 4,316,043; U.S. Pat. No. 4,317,922; U.S. Pat. No. 4,337,354; U.S. Pat. No. 4,356,313; U.S. Pat. No. 4,356,314; U.S. Pat. No. 4,604,396; U.S. Pat. No. 4,675,341; U.S. Pat. No. 4,731,471; U.S. Pat. No. 4,731,472; U.S. Pat. No. 4,782,073; U.S. Pat. No. 4,797,391; U.S. Pat. No. 4,835,313; U.S. Pat. No. 4,605,760; U.S. Pat. No. 4,605,761; U.S. Pat. No. 4,731,470; U.S. Pat. No. 4,769,370; and U.S. Pat. No. 4,777,281, each incorporated herein by reference in its entirety. In some embodiments, fluorenone derivatives for use in compositions and methods described herein are those described in U.S. Pat. No. 6,251,898, incorporated herein by reference in its entirety.

In some embodiments, fluorenone derivatives for use in compositions and methods described herein are analogs (e.g., ether or ester analogs) of R-(+)-(5,6-dichloro-2,3,9,9a-tetrahydro-7-hydroxy-9a-hydrocarbyl-1H-fluoren-3-one compounds having a general chemical structure of Formula (1):

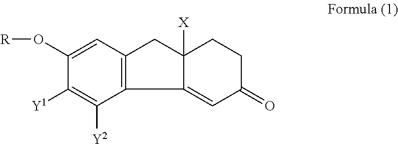

where R, X, and Y¹ and Y² are where X can be a lower alkyl, substituted alkyl, or cycloalkyl group, R can be an ether, ester, or amide group, and Y¹ and Y² can be, independently, halogen, hydrogen, or methyl. More specifically, according to the above formula, R, X, and Y¹ and Y² can be as defined in U.S. Pat. No. 6,251,898, incorporated herein by reference in its entirety.

In one embodiment, the fluorenone derivative is [(R)-(+)-(5,6-dichloro 2,3,9,9a-tetrahydro 3-oxo-9a-propyl-1H-fluoren-7-yl)oxy]acetic acid (DPOFA). According to the general formula above, DPOFA has a propyl group in the R(+) orientation attached to the 9a-position as “X”; the “R” group a carboxymethyl group attached to the 7-carbon atom; and Y¹ and Y² are chorine. The specific structure for DPOFA is as follows:

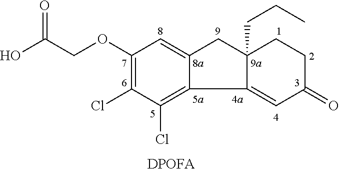

DPOFA is also known as L-644711. DPOFA has been systemically administered to humans in clinical trials for trauma-induced brain edema. DPOFA can inhibit reactive gliosis and facilitate fluid removal, thereby providing effective treatment for ophthalmic conditions, such as retinal detachment and AMD.

It has been reported that DPOFA is an effective inhibitor of glial cell swelling and reactive gliosis in the central nervous system. As shown herein, in an ex vivo tissue culture model, DPOFA effectively stimulates water removal across the retinal pigment epithelium (RPE). Thus, DPOFA and other similar compounds, can effectively remove fluid from subretinal space to choroidal circulation during retinal detachment. Further, down-regulation of gliosis, for example in Muller cells, can increase photoreceptor survival in subjects having or at risk for AMD.

DPOFA can be formulated for local ophthalmic use.

Additional exemplary fluorenone derivatives that can be included in compositions and methods described herein include:
2-{[(5,6-dichloro-2,3,9,9a-tetrahydro-3-oxo-9a-propyl-1H-fluoren-7-yl)oxy]methyl}-tetrahydro-1,3-oxazine;
2-{[(5,6-dichloro-2,3,9,9a-tetrahydro-3-oxo-9a-propyl-1H-fluoren-7-yl)oxy]methyl}oxazoline;
2-{[(5,6-dichloro-2,3,9,9a-tetrahydro-3-oxo-9a-propyl-1H-fluoren-7-yl)oxy]methyl}thiazoline;
5,6-dichloro-9a-propyl-7-(2-pyridylmethoxy)-2,3,9,9a-tetrahydro-1H-fluoren-3-one;
5,6-dichloro-9a-propyl-7-(3-pyridylmethoxy)-2,3,9,9a-tetrahydro-1H-fluoren-3-one;
5,6-dichloro-9a-propyl-7-(4-pyridylmethoxy)-2,3,9,9a-tetrahydro-1H-fluoren-3-one;
5,6-dichloro-2,3,9,9a-tetrahydro-7-[4-(2-oxazolinyl)-phenylmethoxy]-9a-propyl-1H-fluoren-3-one;
5,6-dichloro-2,3,9,9a-tetrahydro-7-[3-(2-oxazolinyl)-phenylmethoxy]-9a-propyl-1H-fluoren-3-one;
5,6-dichloro-2,3,9,9a-tetrahydro-7-[2-(2-oxazolinyl)-phenylmethoxy]-9a-propyl-1H-fluoren-3-one;

enantiomers thereof; and

pharmaceutically acceptable salts thereof.

A compound described herein can be administered as a prodrug.

Formulation

The agents and compositions described herein can be formulated by any conventional manner using one or more pharmaceutically acceptable carriers or excipients as described in, for example, Remington's Pharmaceutical Sciences (Gennaro, editor), 21st edition, ISBN 0781746736 (2005); Ophthalmic Drug Delivery Systems (Mitra, editor), 2d edition, ISBN10 0824741242 (2003); Intraocular Drug Delivery (Jaffe, editor) ISBN10 0824728602 (2006), each incorporated herein by reference in its entirety. Such formulations will contain a therapeutically effective amount of a biologically active agent described herein, preferably in purified form, together with a suitable amount of carrier so as to provide the form for proper administration to the subject.

The formulation should suit the mode of administration. The agents of use with the current invention can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. In various embodiments, agents described herein are formulated for ophthalmic administration. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood and retinal levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

In some embodiments, the pharmaceutical formulation includes hyaluronic acid.

In some embodiments, the pharmaceutical formulation includes silicon oil. Silicon oil is conventionally used in the treatment of retinal detachment, where the oil is injected into the eye and mechanically holds the retina in place until it reattaches.

Compounds described herein can also be used in combination with other therapeutic modalities, as described further below. Thus, in addition to the therapies described herein, one may also provide to the subject other therapies known to be efficacious for treatment of the disease, disorder, or condition.

A compound described herein can be formulated or administered with an ophthalmic drug. Ophthalmic drugs include, but are not limited to, those listed in Ophthalmic Drug Facts, 21^{st} edition, Bartlett, ed., Lippincott Williams & Wilkins, 2009, ISBN10 1574393138. Examples of ophthalmic drugs that can be formulated or administered with a compound described herein include, but are not limited to, dyes, topical anesthetics, mydriatics, cycloplegic mydriatics, anticholinergics, anti-inflammatories, corticosteroids, NSAIDS, artificial tears or lubricants (e.g., carboxymethylcellulose, hydroxypropyl methylcellulose, white petrolatum, mineral oil, lanolin), anti-infectives, antibiotics, antifungals, antivirals, epinephrines, beta blockers, surgical adjuncts, intraocular irrigants, and viscoelastic agents.

For example, an ophthalmic drug that can be formulated or administered with a compound described herein can be selected from Acular (ketorolac tromethamine), AK-Con-A (naphazoline ophthalmic), Akten (lidocaine hydrochloride), Alamast, Alphagan (brimonidine), Alrex, Atropine, Avastin (bevacizumab), AzaSite (azithromycin), Azopt, Bacitracin, Betadine, Betaxolol, Betaxon, Betoptic, Brinzolamide, BSS, Carbachol, Cefazolin, Celluvisc, Chloramphenicol, Ciloxan, Ciprofloxacin, Cosopt, Demecarium, Denufosol tetrasodium, Dexamethasone, Dipivefrin, Dorzolamide, Durezol (difluprednate), Epinephrine, Fluorescein, Flurbiprofen, Gentamicin, Goniosol, Gramicidin, Humorsol, Hylartin, Hypertonic NaCl, Indocycanine Green, Itraconazole, Latanoprost, Lotemax, Lucentis (ranibizumab), Lumigan (bimatoprost ophthalmic solution), Macugen (pegaptanib), Mannitol, Methazolamide, Miconazole, Miostat, Muro 128, Neomycin, Neptazane, Ocuflox, OcuHist, Ofloxacin, Oxytetracycline, Palomid 529, Phenylephrine, Physostimine, Pilocarpine, Plasmin enzyme, Polymyxin B, Prednisolone, Proparacaine, Propine, Puralube, Quixin (levofloxacin), Rescula (unoprostone isopropyl ophthalmic solution), Restasis (cyclosporine ophthalmic emulsion), Rose Bengal, sodium hyaluronate, Suprofen, Terramycin, Timolol, Tobramycin, Triamcinolone, Trifluridine, Tropicamide, Trusopt, Valcyte (valganciclovir HCl), Vidarabine, Vira-A, Viroptic, Vistide (cidofovir injection), Visudyne (verteporfin for injection), Vitrase (hyaluronidase), Vitrasert Implant, Vitravene Injection, Xalatan, and Zaditor.

Therapeutic Methods

Also provided is a process of treating ophthalmic conditions, such as retinal detachment and AMD, or related conditions, in a subject in need. A therapeutically effective amount of a compound described herein can be administered to a subject, so as to treat an ophthalmic condition by inhibiting reactive gliosis or facilitating fluid removal, or both. Treatment methods described herein can decrease the need for surgery, decrease the rate of surgical complications, or reduce the need for repeated surgeries. Given that the standard therapy for retinal detachment is surgery, methods described herein can provide an alternative or an adjunct to an invasive procedure to reattach the retina.

Methods described herein are generally performed on a subject in need thereof. A subject in need of the therapeutic methods described herein can be diagnosed with an ophthalmic condition, such as retinal detachment or AMD, or related conditions, or at risk thereof.

For example, a subject in need can be diagnosed with retinal detachment. As another example, a subject can suffer symptoms of a posterior vitreous detachment. As another example, a subject in need can undergoing a procedure known to increase the incidence of retinal detachment, such as cataract surgery. Examples of conditions related to retinal detachment include, but are not limited to, retinoschisis and chemical or thermal burn and retinal damage due to head trauma (e.g., battlefield injuries).

For example, a subject in need can be diagnosed with AMD. As another example, a subject can be diagnosed as at risk for AMD. A person at risk for AMD can, for example, have one or more of: a family history of AMD, a gene mutation associated with AMD (e.g., mutation in complement system proteins factors H, B, or 3 genes; mutation in ATP synthase gene; mutation in ABD transporter gene, Arg80Gly variant of the complement protein C3, autosomal dominant fibulin-5 mutation), abnormal drusen deposits, hypertension, high cholesterol, obesity, high fat intake, oxidative stress, Caucasian race, light exposure (e.g., blue light exposure), and smoking tobacco.

An effective amount of a compound described herein can inhibit reactive gliosis. An effective amount of a compound described herein can facilitate fluid removal from the retina. An effective amount of a compound described herein can inhibit reactive gliosis and facilitate fluid removal from the retina.

In the case of rhegmatogenous retinal detachment, a defect or tear can be present in the retina. Administration of compounds described herein to a subject diagnosed with rhegmatogenous retinal detachment can aid or accelerate spontaneous, non-surgical healing of a retinal detachment. Administration of compounds described herein to a subject diagnosed with rhegmatogenous retinal detachment can expand the (limited) time window during which surgical correction can be performed. Administration of compounds described herein to a subject diagnosed with rhegmatogenous retinal detachment can occur during a surgical correction procedure. Administration of compounds described herein to a subject diagnosed with rhegmatogenous retinal detachment can after a surgical correction procedure.

In the case of non-rhegmatogenous retinal detachment, usually no tear or defect is present in the retina. Administration of compounds described herein to a subject diagnosed with non-rhegmatogenous retinal detachment can result in fluid removal or prevention of gliosis. Administration of compounds described herein to a subject diagnosed with non-rhegmatogenous retinal detachment can provide additional time to address an underlying systemic illness (such as diabetes). Administration of compounds described herein to a subject diagnosed with non-rhegmatogenous retinal detachment can aid or accelerate spontaneous, non-surgical healing of a retinal detachment.

In the case of atrophic (dry) AMD, RPE abnormalities can lead to secondary degeneration of photoreceptors (e.g., rods and cones) in the macular region (see Petrukhin 2007 Expert Opin. Ther. Targets 11(5), 625-639). Though RPE abnormalities are thought to constitute the primary lesion in atrophic AMD, it is dysfunction and degeneration of photoreceptor cells that can lead to loss of vision. A proportion of AMD patients (about 10-20%) can develop choroidal neovascularization, a form of the disease known as neovascular (wet) AMD, which is associated with the most severe visual loss. In neovascular (wet) AMD, blood vessels can grow up from the choroid behind the retina, which can cause retinal detachment. Death of photoreceptor cells because of atrophy or neovascularization can account for the vision loss in AMD patients. For at least the reasons described above, photoreceptor preservation can provide an effective therapeutic strategy for AMD (e.g., dry AMD).

Muller cells are thought to directly mediate photoreceptor survival (see Zack 2000 Neuron 26(2), 285-286; Harada et al. 2000 Neuron 26(2), 533-541; Wahlin et al. 2000 Invest Ophthalmol V is Sci 41(3), 927-936; Campochiaro et al. 2001 Exp Eye Res 73(5), 693-701). Muller cell abnormalities in the form of reactive gliosis have been documented in human retinas with AMD (see Guidry et al 2002 Invest. Ophthalmol. Vis. Sci. 43(1), 267-273; Lopez et al. 1996 Invest. Ophthalmol. Vis. Sci. 37(5), 855-868; Curcio et al. 1996 Invest. Ophthalmol. Vis. Sci. 37(7), 1236-1249; Madigan et al. 1994 Retina 14(1), 65-74) and retinitis pigmentosa (Fariss et al. Am J Ophthalmol 129(2), 215-223), as well as in animal retinas with different forms of photoreceptor degeneration (RCS rats, see Hartig et al. 1995 J Neurocytol 1995 24(7), 507-1711; F344 rats, see DiLoreto et al. 1995 Brain Res 698(1-2), 1-14; Abyssinian cats, see Ekstrom et al. 1988 Invest Ophthalmol V is Sci 29(9), 1363-71; light-induced, see Grosche et al. Neurosci Lett 185(2), 119-2214; and retinal detachment-induced, see Lewis et al. 1995 Invest Ophthalmol V is Sci 36(12), 2404-16, rod degeneration). Furthermore, down-regulation of reactive gliosis has been shown to be successful for neuroprotection in CNS (see Jones et al. 1999 J Neurobiol 40(4), 560-573). Thus, photoreceptor protection in AMD can be achieved through down-regulation of gliosis (e.g., gliosis in Muller cells) via administration of a compound described herein.

A determination of the need for treatment will typically be assessed by a history and physical exam consistent with the ophthalmic condition, such as retinal detachment or AMD. Diagnosis of the various conditions treatable by the methods described herein is within the skill of the art. The subject can be an animal subject, preferably a mammal, more preferably horses, cows, dogs, cats, sheep, pigs, mice, rats, monkeys, guinea pigs, and chickens, and most preferably a human.

According to the methods described herein, administration can be parenteral, pulmonary, oral, topical, intradermal, intramuscular, intraperitoneal, intravenous, subcutaneous, intranasal, epidural, ophthalmic, buccal, or rectal administration. In various embodiments, administration is ophthalmic. For example, administration can be subscleral, subtenon, subconjanctival, intravitreal, or topical.

In some embodiments, a compound described herein can be delivered to the target tissue (e.g., retina) by intravitreal injections. Intravitreal injection is a standard route for ophthalmic drug delivery. The intravitreal injection of compounds described herein can be accommodated in a brief office procedure and can avoid potential complications of surgery while being similarly, equally, or more efficacious.

In some embodiments, a slow release formulation (e.g., a poly(lactic-co-glycolic acid) formulation) can be administered where sustained delivery to the retina is desired.

When used in the treatments described herein, a therapeutically effective amount of a compound described herein can be employed in pure form or, where such forms exist, in pharmaceutically acceptable salt form and with or without a pharmaceutically acceptable excipient. For example, the compounds of the invention can be administered, at a reasonable benefit/risk ratio applicable to any medical treatment, in a sufficient amount to inhibit reactive gliosis or facilitate fluid removal from the retina.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where large therapeutic indices are preferred.

The specific therapeutically effective dose level for any particular subject will depend upon a variety of factors including the disorder being treated and the severity of the disorder; activity of the specific compound employed; the specific composition employed; the age, body weight, general health, sex and diet of the patient; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Shame, (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present invention will be decided by an attending physician within the scope of sound medical judgment.

Administration of a compound described herein can occur as a single event or over a time course of treatment. For example, a compound described herein can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for an ophthalmic condition, such as retinal detachment or AMD. As discussed above, a compound described herein can be administered as an adjunct to surgical correction of retinal detachment. As another example, a compound described herein can be administered with a drug typically administered via intravitreal injection or having a known side-effect (from the drug or route of administration) of retinal detachment. For example, a compound described herein can be administered in conjunction with gene therapy protocols involving sub-retinal injection, where such procedures can induce retinal detachment. Use of a compound described herein can avoid, in part or in whole, or reduce the occurrence of such side effects.

A fluorenone derivative described herein can be administered or formulated with an ophthalmic drug. Exemplary ophthalmic drugs can be as discussed above (see e.g., Ophthalmic Drug Facts, 21^{St} edition, Bartlett, ed., Lippincott Williams & Wilkins, 2009, ISBN 10 1574393138).

A fluorenone derivative can be administered simultaneously or sequentially with another agent, such as ophthalmic drug, an antibiotic, or an antiinflammatory. For example, a fluorenone derivative can be administered simultaneously with another agent, such as ophthalmic drug, an antibiotic, or an antiinflammatory. Simultaneous administration can occur through administration of separate compositions, each containing one or more of a fluorenone derivative, an ophthalmic drug, an antibiotic, an antiinflammatory, or another agent. Simultaneous administration can occur through administration of one composition containing two or more of a fluorenone derivative, an ophthalmic drug, an antibiotic, an antiinflammatory, or another agent. A fluorenone derivative can be administered sequentially with an ophthalmic drug, an antibiotic, an antiinflammatory, or another agent. For example, a fluorenone derivative can be administered before or after administration of an ophthalmic drug, an antibiotic, an antiinflammatory, or another agent.

Treatment in accord with the methods described herein can include monitoring the subject for the ophthalmic condition of interest. For example, treatment can include monitoring the subject for one or more of: retinal re-detachment, hemorrhage infection, buckle extrusion, lens trauma, cataract progression, and proliferative vitreoretinopathy. As another example, treatment can include monitoring the subject for one or more of: drusen, pigmentary alterations, exudative changes (e.g., hemorrhages in the eye, hard exudates, subretinal/sub-RPE/intraretinal fluid), atrophy (e.g., incipient and geographic), visual acuity drastically decreasing (e.g., two levels or more, such as 20/20 to 20/80), preferential hyperacuity perimetry changes (for wet AMD), blurred vision, central scotomas, distorted vision (i.e., metamorphopsia), difficulty discerning colors, slow recovery of visual function after exposure to bright light, or a loss in contrast sensitivity. As another example, treatment can include monitoring the subject according to an Amsler Grid Test or a contrast sensitivity test. In some embodiments, the method of treatment includes one or more additional administrations of a compound described herein according to results from the monitoring step.

Administration

Compositions described herein can be administered in a variety of means known to the art (see e.g., Ophthalmic Drug Delivery Systems (Mitra, editor), 2d edition, ISBN10 0824741242 (2003); Intraocular Drug Delivery (Jaffe, editor) ISBN10 0824728602 (2006)). As discussed above, administration can be ophthalmic, intraocular, topical, or injection. In some embodiments, administration is intravitreal injection.

Compositions comprising an agent described herein can be administered in a variety of methods well known in the arts. Administration can include, for example, methods involving direct injection (e.g., stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents will be known to the skilled artisan and are within the scope of the invention.

Delivery systems may include, for example, an infusion pump which may be used to administer the agent in a manner similar to that used for delivering insulin or chemotherapy to specific organs or tumors. Typically, using such a system, the agent(s) is administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

A compounds described herein can be encapsulated and administered in a variety of carrier delivery systems (see e.g., Ophthalmic Drug Delivery Systems (Mitra, editor), 2d edition, ISBN10 0824741242 (2003); Intraocular Drug Delivery (Jaffe, editor) ISBN10 0824728602 (2006)). Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for compound delivery can: provide for intracellular delivery; tailor biomolecule/agent release rates; increase the proportion of biomolecule that reaches its site of action; improve the transport of the drug to its site of action; allow co-localized deposition with other agents or excipients; improve the stability of the agent in vivo; prolong the residence time of the agent at its site of action by reducing clearance; decrease the nonspecific delivery of the agent to non-target tissues; decrease irritation caused by the agent; decrease toxicity due to high initial doses of the agent; alter the immunogenicity of the agent; decrease dosage frequency, improve taste of the product; or improve shelf life of the product.

Kits

Also provided are kits. Such kits can include the compositions of the present invention and, in certain embodiments, instructions for administration. Such kits can facilitate performance of the methods described herein. When supplied as a kit, the different components of the composition can be packaged in separate containers and admixed immediately before use. Components include, but are not limited to compounds described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, or sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Compound Effect on Stimulation of Water Transport Across the Bovine RPE-Choroid Complex

The effect of DPOFA was examined in an ex vivo bovine RPE-choroid complex model.

Preparation of solutions.

Ringer's solution contained 120 mM NaCl, 5 mM KCl, 23 mM NaHCO₃, 1 mM MgCl2, 1.8 mM CaCl2, 2.0 mM taurine, and 10 mM glucose at pH 7.4. The osmolariy of the solution was 295±5 mOsM. The solution was placed in the cell culture incubator set at 37° C. with 5% CO₂. The solution was kept in the incubator for at least 2 says before use. Glutathione at final concentration of 1 mM was added to solutions minutes before the use in water transport experiments.

To prepare 5.5 ml of the 108 mM DPOFA stock solution, 220 mg of DPOFA was weighted. Four milliliters of water was added to the drug followed by addition of 104 microliters of 5M NaCl solution. Small magnetic stirring bar was placed to the drug suspension followed by immersion of the pH electrode. Slow addition of 1N NaOH solution began with careful pH monitoring. NaOH was added until pH reached the 7.2-7.4 range at which point the drug was completely dissolved. pH electrode and magnetic stirring bar were removed, and the volume was adjusted to 5.5 ml. The stock was aliquoted and stored at −20° C. Working DPOFA dilutions were made in Ringer's solution or in PBS.

Preparation of Ussing-type chambers for water transfer experiments.

An Ussing-type chamber was used to assess water transport across tissues preparations. Three hours before the experiment two Ussing-like chambers were assembled and filled with phosphate buffered saline. The cambers were placed on the thermo-jacketed stand to pre-warm the chambers to the 37° C.

Preparation of the bovine RPE-choroid circular tissue sheets for water transport experiments.

Bovine eyes were obtained from the local abattoir and placed to the cold Ringer's solution containing 120 mM NaCl, 5 mM KCl, 23 mM NaHCO3, 1 mM MgCl2, 1.8 mM CaCl2, 2.0 mM taurine, and 10 mM glucose pH 7.4. Eyes were transferred to the lab within 2-3 hours after enucleation and rinsed repeatedly in Ringer's solution. Excessive muscle and connective tissue were trimmed at room temperature. The enucleated eye was dissected at pars plana, and the anterior portion was discarded along with the vitreous. If sensory retina detached during the removal of vitreous, the eye was considered unusable and was discarded. After the removal of vitreous, the posterior portion of the eye with the attached neuroretina was visually examined to locate the area not containing large blood vessels in the sclera. Using a size 13 brass cork bore, the selected area was punched through using a hammer.

The resulting tissue “button” was placed in a Petri dish containing Ringer's solution. The neuroretina was carefully peeled off and discarded. The flat circular sheet of the RPE-choroid tissue was carefully removed with forceps, placed on the flat spoon and transported to the Petri dish where the “basal” round window of the Ussing-type chamber (containing metal mesh) was placed and covered with Ringer's solution. The choroid-RPE circular sheet was placed on the metal mesh of the“basal” window with choroid side down. The RPE side of the RPE-choroid sheet was covered with the nylon mesh shown, followed by clipping with the “apical” portion of the Ussing-type chamber window.

The pre-warmed Ussing-like chambers were removed from the thermo-jacketed stand, freed from the PBS solution, and disassembled. The assembled chamber window with the inserted RPE-choroid circular tissue sheet was installed in the chamber. The assembled Ussing-like chambers containing the RPE-choroid tissue were placed back to the thermo-jacketed stand.

Pre-warmed Ringer's solution was added first to the apical side and then to the basal side of the chamber. The temperature within the chambers was checked and if it reached 35° C., the electrical resistance was measured using the modified electrode and the DVC-1000 Voltage/Current Clamp instrument (WPI, Inc.). If resistance of the tissue was less than 100Ω, the preparation was discarded and a new tissue circle was inserted into the camera. If resistance was more than 100Ω, the tissue preparation was considered normal. Two cylinders containing measuring capillaries were inserted inside the chamber starting from the apical side and then to the basal side. The levels of the liquid in basal and apical baths were adjusting using a long barrel syringe needle.

A horizontal observation microscope with objective grades was used to measure water transport by tracing changes in positions of the meniscus in each of the two capillaries. Recordings of the meniscus position were started shortly after adjusting the liquid levels. DPOFA was added at different concentration to the Ussing-like chamber baths 1-3 hours after beginning of the meniscus level measurements. The upward shift in the basal capillary meniscus position in response to addition of DPOFA was expressed in micrometers of increase in meniscus level when compared to meniscus position at time zero. Upward movement of liquid in the basal side capillary reflected the increase in the pumping rate from apical to basolateral side of the RPE-choroid preparation in response to DPOFA addition.

Results showed that DPOFA stimulated water transport across the bovine RPE-choroid complex.

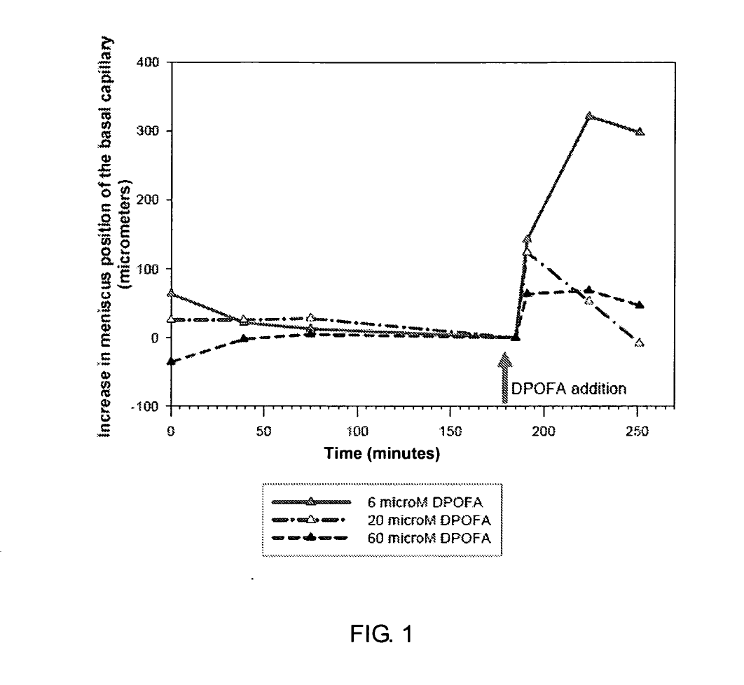

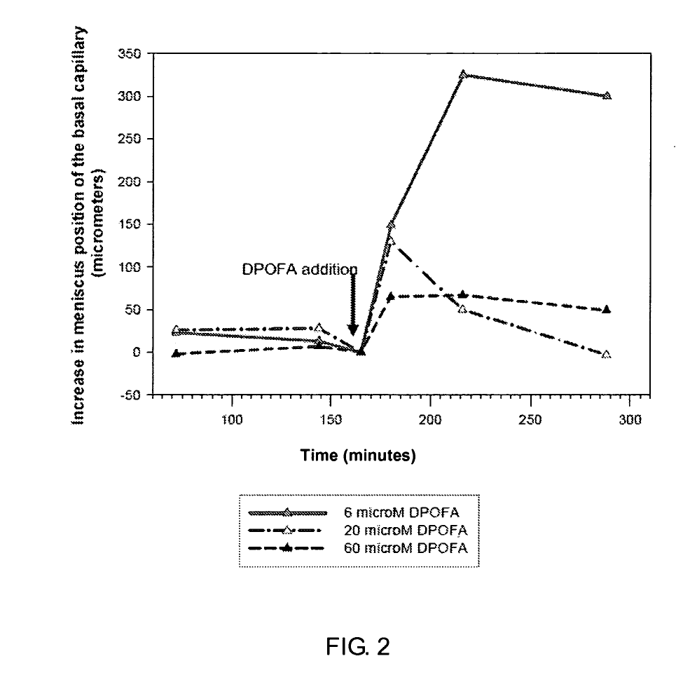

Exemplary data showing the effect of DPOFA on stimulation of water transport from apical to the basal side of the RPE-choroid complex are presented in shown FIG. 1 and FIG. 2. In a first experiment, addition of DPOFA stimulated significant increase in transport of water from apical to basal side of the RPE-choroid complex as can be judged by the upward shift of the meniscus position in a capillary positioned in the basal half of the Ussing-like chamber (see e.g., FIG. 1). 6 μM concentration of DPOFA induced more significant transport of water than 20 μM and 60 μM drug concentrations. In a second experiment, DPOFA increased the movement of water from apical to basal side of the RPE-choroid complex (see e.g., FIG. 2). The strongest stimulation of water transport was seen at 6 μM concentration.

The data presented herein shows that DPOFA stimulates movement of water from the apical side of the RPE-choroid complex to its basal side. The apical side of the RPE corresponds to the subretinal space of the retina, indicating that DPOFA is effective in movement of water from the subretinal space, thus inducing resolution of retinal detachment.

1. A method for treating an ophthalmic condition comprising:
administering to a subject in need thereof a pharmaceutical composition comprising an effective amount of a compound of formula (1) 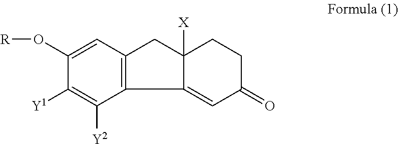
wherein
X is selected from the group consisting of lower alkyl containing 1 to 3 carbon atoms; substituted lower alkyl; and lower cycloalkyl;
R is a substituted alkyl group in which the substituents are selected from the group consisting of aryl and substituted aryl; and substituted or unsubstituted heterocyclic rings having 0 or 1 nitrogen atom and at least one double bond wherein the alkyl group is attached to a carbon atom of the heterocyclic ring;
Y¹ and Y² are independently selected from the group consisting of halogen, hydrogen, and methyl; and
the ophthalmic condition is selected from the group consisting of retinal detachment or age-related macular degeneration (AMD).

2. The method according to claim 1, wherein X is selected from the group consisting of propyl, hydroxyethyl, haloethyl, and cycloalkyl having less than 6 carbons.

3. The method according to any one of claims 1-2 wherein R is a heterocyclic-alkyl group.

4. The method according to any one of claims 1-3 wherein R is an oxazinyl-alkyl group.

5. The method according to claim 3 wherein the compound is selected from the group consisting of:
2-{[(5,6-dichloro-2,3,9,9a-tetrahydro-3-oxo-9a-propyl-1H-fluoren-7-yl)oxy]methyl}-tetrahydro-1,3-oxazine;
2-{[(5,6-dichloro-2,3,9,9a-tetrahydro-3-oxo-9a-propyl-1H-fluoren-7-yl)oxy]methyl}oxazoline;
2-{[(5,6-dichloro-2,3,9,9a-tetrahydro-3-oxo-9a-propyl-1H-fluoren-7-yl)oxy]methyl}thiazoline;
enantiomers thereof; and
pharmaceutically acceptable salts thereof.

6. The method according to any one of claims 1-3 wherein R is a pyridyl-alkyl group.

7. The method according to claim 6 wherein the compound is selected from the group consisting of:
5,6-dichloro-9a-propyl-7-(2-pyridylmethoxy)-2,3,9,9a-tetrahydro-1H-fluoren-3-one;
5,6-dichloro-9a-propyl-7-(3-pyridylmethoxy)-2,3,9,9a-tetrahydro-1H-fluoren-3-one;
5,6-dichloro-9a-propyl-7-(4-pyridylmethoxy)-2,3,9,9a-tetrahydro-1H-fluoren-3-one;
enantiomers thereof; and
pharmaceutically acceptable salts thereof.

8. The method according to any one of claims 1-2 wherein R is a heterocyclicaralkyl group.

9. The method according to claim 8 wherein the compound is selected from the group consisting of:
5,6-dichloro-2,3,9,9a-tetrahydro-7-[4-(2-oxazolinyl)-phenylmethoxy]-9a-propyl-1H-fluoren-3-one;
5,6-dichloro-2,3,9,9a-tetrahydro-7-[3-(2-oxazolinyl)-phenylmethoxy]-9a-propyl-1H-fluoren-3-one;
5,6-dichloro-2,3,9,9a-tetrahydro-7-[2-(2-oxazolinyl)-phenylmethoxy]-9a-propyl-1H-fluoren-3-one; and
pharmaceutically acceptable salts thereof.

10. The method according to claim 1, wherein X is propyl; R is carboxymethyl; and Y¹ and Y² are chorine.

11. The method according to claim 1, wherein the compound is [(R)-(+)-(5,6-dichloro 2,3,9,9a-tetrahydro 3-oxo-9a-propyl-1H-fluoren-7-yl)oxy]acetic acid (DPOFA), having a structure as follows:

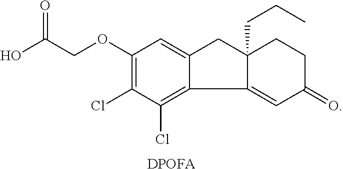

12. The method according to claim 1, wherein the compound is a prodrug.

13. The method according to any one of claims 1-12, wherein the subject is a mammal.

14. The method according to any one of claims 1-13, wherein the subject is selected from the group consisting of a human, monkey, horse, cow, dog, cat, sheep, pig, mice, rat, guinea pig, and chicken.

15. The method according to any one of claims 1-14, wherein the composition is administered ophthalmically.

16. The method according to any one of claims 1-15, wherein the administration comprises subscleral, subtenon, subconjanctival, intravitreal, or topical administration.

17. The method according to any one of claims 1-15, wherein the administration comprises intravitreal injection.

18. The method according to any one of claims 1-17, wherein the composition further comprises an ophthalmic agent selected from the group consisting of an ophthalmic dye, an ophthalmic anesthetic, an ophthalmic mydriatic, an ophthalmic cycloplegic mydriatic, an ophthalmic anticholinergic, and ophthalmic anti-inflammatory, an ophthalmic corticosteroid, ophthalmic artificial tears or lubricants, an ophthalmic antibiotic, an ophthalmic antifungal, an ophthalmic antiviral, an ophthalmic epinephrine, an ophthalmic beta blocker, an ophthalmic surgical adjunct, an ophthalmic intraocular irrigant, and an ophthalmic viscoelastic agent.

19. The method according to claim 18, wherein the ophthalmic agent is selected from the group consisting of: Acular (ketorolac tromethamine), AK-Con-A (naphazoline ophthalmic), Akten (lidocaine hydrochloride), Alamast, Alphagan (brimonidine), Alrex, Avastin (bevacizumab), Atropine, AzaSite (azithromycin), Azopt, Bacitracin, Betadine, Betaxolol, Betaxon, Betoptic, Brinzolamide, BSS, Carbachol, Cefazolin, Celluvisc, Chloramphenicol, Ciloxan, Ciprofloxacin, Cosopt, Demecarium, Denufosol tetrasodium, Dexamethasone, Dipivefrin, Dorzolamide, Durezol (difluprednate), Epinephrine, Fluorescein, Flurbiprofen, Gentamicin, Goniosol, Gramicidin, Humorsol, Hylartin, Hypertonic NaCl, Indocycanine Green, Itraconazole, Latanoprost, Lotemax, Lucentis (ranibizumab), Lumigan (bimatoprost ophthalmic solution), Macugen (pegaptanib), Mannitol, Methazolamide, Miconazole, Miostat, Muro 128, Neomycin, Neptazane, Ocuflox, OcuHist, Ofloxacin, Oxytetracycline, Palomid 529, Phenylephrine, Physostimine, Pilocarpine, Plasmin enzyme, Polymyxin B, Prednisolone, Proparacaine, Propine, Puralube, Quixin (levofloxacin), Rescula (unoprostone isopropyl ophthalmic solution), Restasis (cyclosporine ophthalmic emulsion), Rose Bengal, sodium hyaluronate, Suprofen, Terramycin, Timolol, Tobramycin, Triamcinolone, Trifluridine, Tropicamide, Trusopt, Valcyte (valganciclovir HCl), Vidarabine, Vira-A, Viroptic, Vistide (cidofovir injection), Visudyne (verteporfin for injection), Vitrase (hyaluronidase), Vitrasert Implant, Vitravene Injection, Xalatan, and Zaditor.

20. The method according to any one of claims 1-19, wherein the pharmaceutical composition comprises a pharmaceutically acceptable carrier or excipient.

21. The method according to any one of claims 1-20, wherein the pharmaceutical composition comprises hyaluronic acid.

22. The method according to any one of claims 1-21, wherein the pharmaceutical composition comprises silicon oil.

23. The method according to claim 1, wherein the ophthalmic condition is retinal detachment.

24. The method according to any one of claims 1-23, further comprising monitoring the subject for one or more of: retinal re-detachment, hemorrhage infection, buckle extrusion, lens trauma, cataract progression, and proliferative vitreoretinopathy.

25. The method according to claim 1, wherein the ophthalmic condition is AMD.

26. The method according to claim 25, wherein the ophthalmic condition is atrophic AMD or neovascular AMD.

27. The method according to claim 26, wherein the ophthalmic condition is atrophic AMD.

28. The method according to claim 26, wherein the ophthalmic condition is neovascular AMD.

29. The method according to any one of claims 1-28, further comprising monitoring the subject for one or more of: drusen, pigmentary alterations, exudative changes, atrophy, decreased visual acuity, preferential hyperacuity perimetry changes, blurred vision, central scotomas, metamorphopsia, difficulty discerning colors, slow recovery of visual function after exposure to bright light, or a loss in contrast sensitivity.

30. The method according to any one of claim 24 or 29, further comprising re-administering the composition.

31. A pharmaceutical composition comprising:
a compound of formula (1) 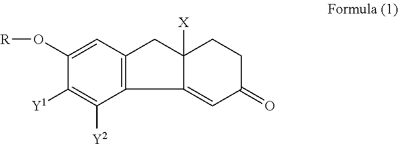
wherein
X is selected from the group consisting of lower alkyl containing 1 to 3 carbon atoms; substituted lower alkyl; and lower cycloalkyl;
R is a substituted alkyl group in which the substituents are selected from the group consisting of aryl and substituted aryl; and substituted or unsubstituted heterocyclic rings having 0 or 1 nitrogen atom and at least one double bond wherein the alkyl group is attached to a carbon atom of the heterocyclic ring; and
Y¹ and Y² are independently selected from the group consisting of halogen, hydrogen, and methyl;
an ophthalmic agent selected from the group consisting of an ophthalmic dye, and ophthalmic anesthetic, an ophthalmic mydriatic, an ophthalmic cycloplegic mydriatic, an ophthalmic anticholinergic, and ophthalmic anti-inflammatory, an ophthalmic corticosteroid, ophthalmic artificial tears or lubricants, an ophthalmic antibiotic, an ophthalmic antifungal, an ophthalmic antiviral, an ophthalmic epinephrine, an ophthalmic beta blocker, an ophthalmic surgical adjunct, an ophthalmic intraocular irrigant, and an ophthalmic viscoelastic agent; and
a pharmaceutically acceptable carrier or excipient.

32. The pharmaceutical composition of claim 31, wherein the compound is [(R)-(+)-(5,6-dichloro 2,3,9,9a-tetrahydro 3-oxo-9a-propyl-1H-fluoren-7-yl)oxy]acetic acid (DPOFA), having a structure as follows:

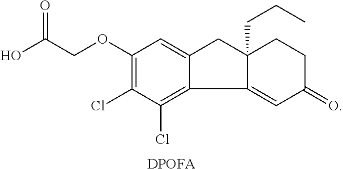

33. The pharmaceutical composition of any one of claims 31-32, wherein the ophthalmic agent is selected from the group consisting of: Acular (ketorolac tromethamine), AK-Con-A (naphazoline ophthalmic), Akten (lidocaine hydrochloride), Alamast, Alphagan (brimonidine), Alrex, Atropine, Avastin (bevacizumab), AzaSite (azithromycin), Azopt, Bacitracin, Betadine, Betaxolol, Betaxon, Betoptic, Brinzolamide, BSS, Carbachol, Cefazolin, Celluvisc, Chloramphenicol, Ciloxan, Ciprofloxacin, Cosopt, Demecarium, Denufosol tetrasodium, Dexamethasone, Dipivefrin, Dorzolamide, Durezol (difluprednate), Epinephrine, Fluorescein, Flurbiprofen, Gentamicin, Goniosol, Gramicidin, Humorsol, Hylartin, Hypertonic NaCl, Indocycanine Green, Itraconazole, Latanoprost, Lotemax, Lucentis (ranibizumab), Lumigan (bimatoprost ophthalmic solution), Macugen (pegaptanib), Mannitol, Methazolamide, Miconazole, Miostat, Muro 128, Neomycin, Neptazane, Ocuflox, OcuHist, Ofloxacin, Oxytetracycline, Palomid 529, Phenylephrine, Physostimine, Pilocarpine, Plasmin enzyme, Polymyxin B, Prednisolone, Proparacaine, Propine, Puralube, Quixin (levofloxacin), Rescula (unoprostone isopropyl ophthalmic solution), Restasis (cyclosporine ophthalmic emulsion), Rose Bengal, sodium hyaluronate, Suprofen, Terramycin, Timolol, Tobramycin, Triamcinolone, Trifluridine, Tropicamide, Trusopt, Valcyte (valganciclovir HCl), Vidarabine, Vira-A, Viroptic, Vistide (cidofovir injection), Visudyne (verteporfin for injection), Vitrase (hyaluronidase), Vitrasert Implant, Vitravene Injection, Xalatan, and Zaditor.
